(19) United States
(12) Patent Application Publication
(10) Pub. No.: US 2013/0079629 A1
(43) Pub. Date: Mar. 28, 2013

(54) PASSIVE, NONINVASIVE TOMOGRAPHY

(71) Applicant: James U. Lemke, La Jolla, CA (US)

(72) Inventor: James U. Lemke, La Jolla, CA (US)

(73) Assignee: James U. Lemke, La Jolla, CA (US)

(21) Appl. No.: 13/242,186

(22) Filed: Sep. 23, 2011

(51) Int. Cl. A61B 6/00

(52) U.S. Cl. 600/425

(57) ABSTRACT

A passive, noninvasive tomography apparatus and method is disclosed for in-depth tissue imaging and lesion detection. In one particular approach, the disclosed apparatus and method is adapted for use in breast imaging.

### BACKGROUND OF THE DISCLOSURE

The present disclosure is directed towards the field of medical diagnostic instruments, and more particularly, to the field of non-invasive, passive imaging for lesion detection through focused microwave radiometry with coherent detection. One particular application of the present disclosure is to the imaging of breast tissue.

Currently, mammography along with physical breast examination is the modality of choice for screening for early breast cancer. Ultrasound, ductography, positron emission mammography (PEM), and magnetic resonance imaging are adjuncts to mammography. Ultrasound is typically used for further evaluation of masses found on mammography or palpable masses not seen on mammograms. Ductograms are still used in some institutions for evaluation of bloody nipple discharge when the mammogram is non-diagnostic. MRI can be useful for further evaluation of questionable findings as well as for screening pre-surgical evaluation in patients with known breast cancer to detect any additional lesions that might change the surgical approach, for instance from breast-conserving lumpectomy to mastectomy. New procedures, not yet approved for use in the general public, including breast tomosynthesis may offer benefits in years to come.

Conventional mammography relies upon detecting the absorption of introduced X-ray radiation by lesions in breast tissue and comparing that absorption to the absorption by normal tissue. That is, mammography is the process of using low-dose amplitude X-rays to examine the human breast and is used as a diagnostic and a screening tool. The goal of mammography is the early detection of breast cancer, typically through detection of characteristic masses and/or microcalcifications. Mammography is believed to reduce mortality from breast cancer. Like all X-rays, mammograms use doses of ionizing radiation to create images. Radiologists then analyze the image for any abnormal findings. It is normal to use longer wavelength X-rays (typically Mo—K) than those used for radiography of bones. Unfortunately, breast compression is necessary in a mammogram procedure, and the same can be rather painful. Moreover, repeated exposures to X-rays can be detrimental to the health of the patient. In fact, overexposure can actually lead to the formation of cancer cells.

Sensitivity and selectivity can also be compromised by this method since non-lesions such as dense tissue, calcifications, and scar tissue have high absorptivity and mask the areas of interest. Typically, lesions have a diameter of about 1 cm when they are detected by mammography and have been growing for 8 to 12 years. In fact, False Negatives of up to 20% and False Positives of about 10-15% are not uncommon. The False Positives frequently lead to invasive procedures such as biopsy, the majority of which find non-malignancy. Moreover, conventional mammography carries the burden of increased probability of inducing cancer with each exposure.

All matter above absolute zero temperature radiates electromagnetic energy in the microwave region at a rate that is accurately modeled by the Rayleigh-Jeans Radiation Law. By detecting this radiation and comparing the temperature of its radiating tissue to that of adjacent volumes of tissue, early indications of cancer are feasible.

Infrared thermography, thermal imaging, and thermal video are examples of infrared imaging science. Thermal imaging cameras detect radiation in the infrared range of the electromagnetic spectrum (roughly 780 nm and longer) and produce surface images of that radiation, called thermograms. However, thermography is limited to essentially surface measurement since the IR penetration is shallow in tissue.

Microwave radiometry is a promising technology for use in medical diagnosis. It is completely passive since no radiation is introduced, but, rather, the natural electromagnetic radiation of a lesion at its elevated temperature is measured in the microwave region (Rayleigh-Jeans Law) and compared with the temperature of adjacent regions of the sample. Lesions experience mitosis at a more rapid rate than host cells and exhibit relatively elevated temperatures, about 1°-3° C., on average. Lesions generate supplemental vascular systems to satisfy their increased metabolic rate (angiogenesis). Differential temperature is a better indicator of increased metabolic rate than is the presence of an X-ray absorber. Absolute temperature is not required to be known; differential temperature relative to adjacent detection cells is adequate to indicate areas of interest.

However, prior attempts at microwave radiometry were limited to surface detection and low resolution due to the very large attenuation of high-frequency microwaves in tissue, distortions induced by the heterogeneous dielectric properties of the surrounding tissue, and the lack of a suitable focusing antenna.
“- - - Although not as widely reported, the use of microwave radiometry as a noninvasive, passive technique for the early detection of cancer appears promising. Wider acceptance of these methods, however, awaits fundamental improvements in the ability to focus energy at depth in human tissue, an important and nontrivial antenna problem”, K. L. Carr, December 1989, revised Aug. 6, 2002, IEEE Transactions on Microwave Theory and Techniques.

Further, an approach characterized as Correlation Microwave Thermography (Introduction to correlation microwave thermography, Mamouni et al., 1983 September; 18 (3): 285-93, J Micro Power) has also been found to be lacking Whereas the approach endeavors to improve localization of thermal gradients in tissues, there is a lack of focusing of the probes employed which results in an inability to differentiate between tissue volumes to an extent necessary to detect small lesions. Thus, a true coherent detection is not achieved by such prior approaches.

Accordingly, what is needed is innovative performance enhancements that will facilitate interpretation of tissue scans including: no breast compression, full 3-D tomographic thermal image of each breast with false color highlighting regions measured at more than 0.1° above the adjacent host tissue, and a detection threshold currently predicted to be about 3 mm in diameter. It is also desirable to have an approach that has associated therewith no X-rays or external emanations of any sort so that a radiation-hardened facility is not required and the health risk obviated. The device should also be comprised of relatively inexpensive electronics and electro-mechanical components so that there is a favorable gross margin at a reduced equipment cost relative to conventional mammogram machines. It should be further possible that the approach may be repeated as often as desired to follow a suspected virulent lesion without any fear of harm to the patient.

The present application addresses these and other needs.

### SUMMARY

Briefly and in general terms, the present disclosure is directed toward a system and method for detecting a lesion within tissue. The disclosure also contemplates treating tissue lesions.

In one particular aspect, the present disclosure is applicable to the identification and treatment of lesions found in breast tissue. The system can define a radiometer that passively measures electromagnetic energy. The radiometer can be a microwave radiometer that measures energy emitted at microwave wavelengths. In particular, the system can be configured to characterize the temperatures of discrete volumes of tissue.

In one approach, the system is embodied in a pair of ellipsoidal antenna assemblies having ellipsoidal partial reflectors with aligned exterior focal points. It is also contemplated that the system can include two or more antenna assemblies including reflectors with conjugate foci. In one particular respect, the reflectors can be ellipsoidal half-reflectors, and can be arranged generally orthogonally. The system is manipulatable so that the common exterior focal point is translated through a volume of tissue. Electromagnetic radiation of the tissue is propagated to internal focal points of each of first and second ellipsoid reflectors. The electromagnetic radiation can then be analyzed to identify differential temperatures of tissue volumes being studied. Tissue cells having elevated temperatures are identified and studied further. The system can be additionally configured to direct energy at the identified lesions for treatment purposes or to ablate the lesion.

The system can further include a first ellipsoidal antenna assembly including a first antenna and a second ellipsoidal antenna assembly including a second antenna. The first and second antennas are positioned at the interior focal points of the first and second reflectors, respectively. The second antenna can be translatable with respect to the first antenna while maintaining a juxtapositional relationship between the first and second ellipsoidal antenna assemblies in other respects. In one embodiment, the second antenna can be configured on a periphery of a turntable so that rotation of the turntable positions the second antenna along a predetermined path. An offset of the second antenna with respect to the first antenna functions to facilitate compensating for varying paths through which electromagnetic energy travels from the tissue site being examined and the detecting first and second antennas. That is, an offset of an interior antenna site can cause or compensate for an offset of an external focal point. Thus, the effective positions of the exterior focal points of the first and second antennas can help ensure a maximum coupling occurs between the antennas. Such a maximum coupling can be detected through impedance measurements. The angular position for peak coupling is noted and employed during subsequent scanning of tissue.

The present system can also include structure and functionality to minimize scanning volumes. In one embodiment, the tissue to be scanned is placed within a receptacle. The receptacle can include a surface characterized by alternating black and white sections, or other contrasting colors. A first antenna assembly can be equipped with a light source and also, a photo detector positioned at the interior focal point. Light energy from the light source is projected onto the surface of the receptacle and the photo detector is employed to measure a net resistance of a focused image, the minimum resistance detected being associated with an in focus image. The location of these minimum resistance points correlate to the contour of the receptacle containing the tissue to be scanned. In this way, the scanning time can be minimized by limiting the scanning volume of the tissue at issue.

In a related method, a preliminary scan can be first conducted for the purpose of locating and mapping the surface of the tissue being examined, and to identify its boundaries. The volume of the tissue being examined is then scanned while locating and storing a position of a second antenna offset when there is a coincidence of external foci of the first and second antenna assemblies. Next, a scan is conducted to identify temperature differentials between tissue sub-volumes being studied. A tomographical 3-D image of the tissue examined can then be generated to thereby highlight possible locations of lesions within the target tissue. In certain circumstances, the system can thereafter be configured to generate and project energy directed at a lesion for treatment purposes.

Other features and advantages of the present invention will become apparent from the following detailed description, taken in conjunction with the accompanying drawings, which illustrate, by way of example, the principles of the invention.

### BRIEF DESCRIPTION OF THE DRAWINGS

FIG. 1 is a graphical representation, depicting a relationship between thermal and anatomical changes in tissue;

FIG. 2 is a graphical representation depicting a relationship between tumor volume and heat production;

FIG. 3 is a schematic representation, depicting a scanning assembly employing a plurality of ellipsoidal antenna assemblies arranged adjacent a tissue receptacle;

FIGS. 4A and 4B are perspective views, depicting an ellipsoidal antenna assembly mounted to a three axis transport assembly;

FIG. 5 is a flow chart, depicting one contemplated approach to tissue scanning;

FIG. 6 is a plotted curve, depicting light levels versus resistance;

FIG. 7 is another plotted curve, depicting a focus quality versus resistance;

FIG. 8 is a schematic representation, depicting a unit cell of volume of tissue to be scanned; and

FIG. 9 is a schematic representation, depicting one tissue scanning approach.

### DETAILED DESCRIPTION OF THE PREFERRED EMBODIMENTS

Referring now to the drawings, which are provided by way of example and not limitation, there is shown a system for detecting lesions within biological tissue. The disclosed system is applicable to various locations of the body. In one particular application, the system can be employed to detect and treat lesions found in breast tissue.

The presence of an elevated temperature in a small volume of tissue relative to the temperature of adjacent volumes of tissue can be an indication of a malignant lesion. The absolute temperature of a sample is of less interest than the local differential temperature. Since all matter above absolute zero Kelvin temperature radiates energy, it is possible, in principle, to measure that temperature through remote sensing. The energy-density of radiation in the microwave region can be accurately modeled by the Rayleigh-Jeans Law. However, since human tissue has conductivity and dielectric properties differing from free space, there are sizable attenuations of signal strength and refractions when regions deep within the tissue are being examined. Consequently, passive microwave radiometry has not been successful previously except for exploring very shallow depths within tissue.

Passive and non-invasive breast cancer detection can be accomplished by a scanning microwave radiometer. As stated, one particular application would be to the detection of breast cancer. Since lesions experience a more rapid rate of mitosis than normal tissue and exhibit angiogenesis, they have relatively elevated temperatures that can indicate the presence of lesions. The Rayleigh-Jeans Radiation Law can be employed to predict accurately the radiation as a function of temperature at microwave frequency. That temperature can be measured in small volumes and compared to the temperature of adjacent small volumes.

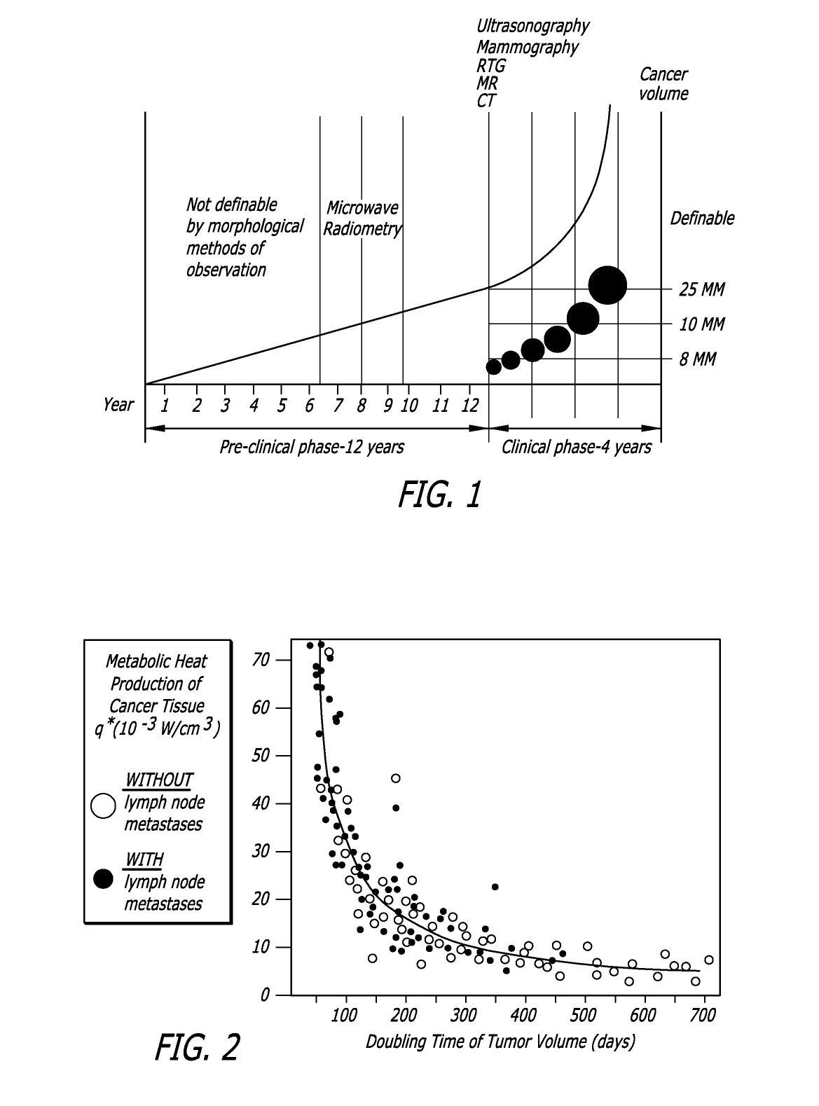

It has been noted that the resting metabolic rate for humans is about 1 mW/cm³. Further, it has been shown that the specific heat production rate associated with lesion growth increases with the doubling time of tumor volume (See FIGS. 1 and 2). A tumor that doubles in volume in two years has a metabolic rate of about 5 mW/cm³ and a corresponding increased temperature of about 1° C.; a tumor that doubles in 60 days has a metabolic rate of about 40 mW/cm³. The corresponding higher temperature of the more aggressive lesions makes microwave radiometry a desirable mode of detection, particularly where scanning procedures can be performed at frequent intervals to monitor the progress of the more virulent lesions without the risk of repeated X-ray exposure.

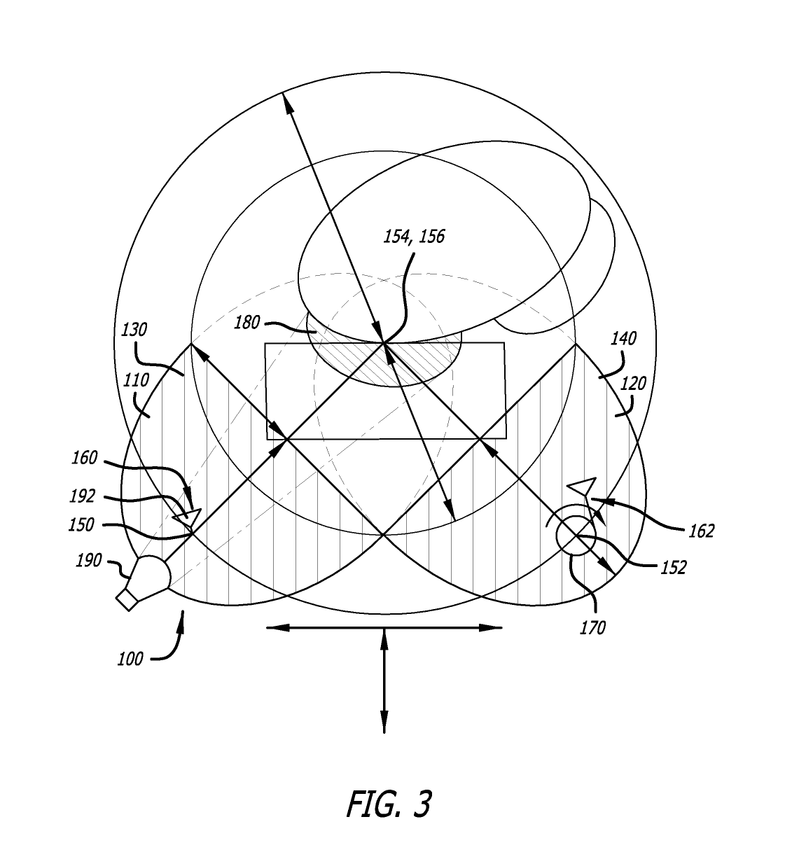

With reference to FIG. 3, there is shown one approach to a passive tissue scanning system. The system can be embodied in an antenna assembly 100 which can be configured to define a radiometer that passively measures electromagnetic energy. The radiometer can be a microwave radiometer that measures energy emitted at microwave wavelengths. The system can further be configured to characterize temperature differences between separate cell units of tissue.

Unfortunately, a single ½-reflector does not have sufficient signal-to-noise ratio (SNR) to differentiate temperature of the tissue of interest from normal tissue with approximately the same temperature. The unwanted signal is considered “clutter” in radar parlance. The solution to this problem lies in employing two ½-reflectors with independent antennas. Various angles between the reflectors are contemplated as is the use of three or more reflectors. By utilizing coherent detection it is possible to eliminate all signals emanating from tissue outside the common focus region. Very large improvements in SNR can be realized by this technique with signals becoming detectable well below the noise floor.

Thus, as shown in FIG. 3, the antenna system 100 includes a first ellipsoidal antenna assembly 110 and a second ellipsoidal antenna assembly 120. Each of the ellipsoidal antenna assemblies 110, 120 include an ellipsoidal reflector 130, 140. As will be developed further below, the ellipsoidal reflectors 130, 140 each have associated therewith, an interior or internal focal point 150, 152 and an exterior or external focal point, respectively 154, 156.

Mounted within the first ellipsoidal reflector 130 is a first antenna 160. The first antenna is positioned at the interior focal point 150 of the first ellipsoidal reflector 130. Mounted at or near the interior focal point 152 of second reflector 140 is a second antenna 162. The second antenna can be asymmetrically affixed to a rotating structure 170, such as a turntable, positioned at the interior focal point 152 of the second reflector 140. In this way, as the turntable rotates, the second antenna 162 will move through a defined path.

The system 100 further includes a tissue receptacle 180. The receptacle 180 can be sized and shaped to receive various tissues, but as shown in FIG. 3, one application can be configured to receive breast tissue. The receptacle is also contemplated to define structure which is flexible but capable of securely retaining tissue for examination. The receptacle 180 is intended to remain stationary while the scanning system 100 moves to complete a scan.

Further, mounted within the first reflector 130 can be a light source 190 and a photocell or photo detector 192. The light source 190 is configured to project light energy onto an exterior of receptacle 180. The photo detector 192 receives light energy reflected off of the receptacle.

In one embodiment, the reflectors 130, 140 define dual ellipsoidal half-reflectors, that are nominally orthogonal with independent antennas 160, 162 located at their respective internal foci 150, 152 and with common external foci 154, 156. Other angles between the reflectors 130, 140 are also contemplated as are other partial ellipsoidal sections. Here, a half-ellipsoidal shape is employed to maximize the opening to an internal cavity defined by the reflectors. The generally orthogonal angle is selected also to enhance operational characteristics.

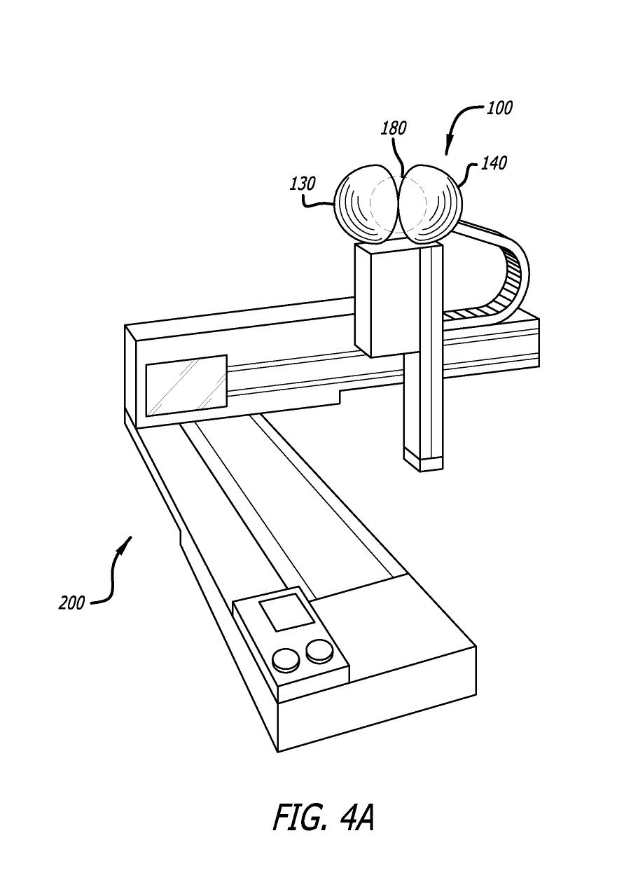

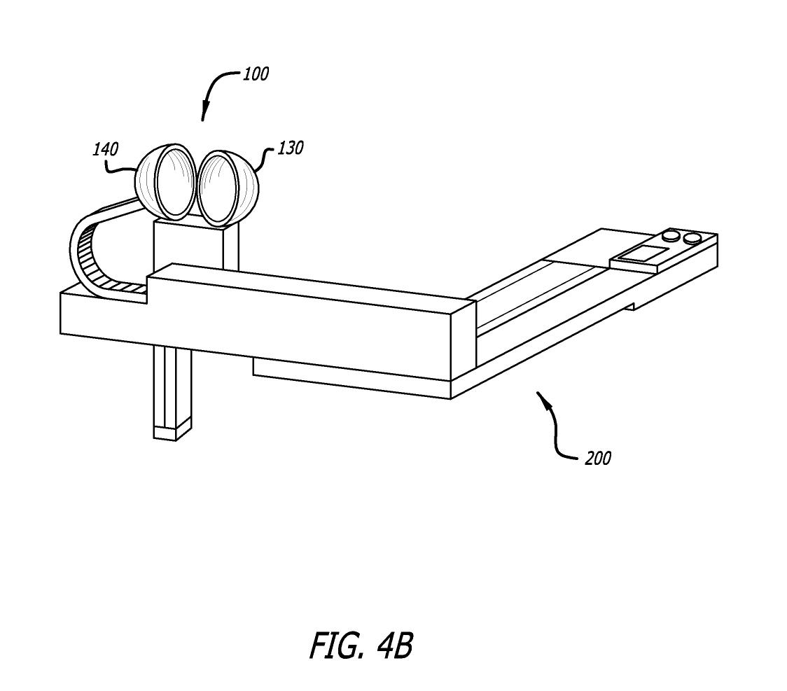

As shown in FIGS. 4A and 4B, the scanning system 100 can be mounted to a three dimensional mechanical stage or transport assembly 200. Although any mounted staging can be employed, one such 3-D stage can be a modified form of an EPSON three axis module. Such an arrangement would permit raster scanning of external common foci 154, 156 of the reflectors 130, 140 positioned within a volume of tissue held with a receptacle 180.

The external focal positions 154, 156 of each reflector 130, 140 may be altered by the differential dielectric properties of the intervening tissue each signal encounters as it travels to its respective antenna so that only an approximate coincidence of external focus may occur. The effective position of the external focal point 156 of a second reflector 140 is “dithered” in the “x” and “z” directions by the turntable 170 to seek coincidence with the external focal point of the first antenna 154 through the mechanical displacement of the second antenna 162 within its reflector 140. By measuring the varying impedance of the coupled antennas during the raster scan due to the “dithered” position of the second antenna 162, the instantaneous position of the second antenna 162 within its reflector 140 that corresponds to peak coupling of the antennas 160, 162 as a function of the “x”, “y”, and “z” position of the first antenna 160 is stored in a “lookup” table for subsequent positioning of the second antenna 162. By this method, coincidence, or near-coincidence, of the common focal points 154, 156 is assured resulting in maximum sensitivity of the correlation process independent of the dielectric properties of the tissue involved. A resonant cavity tuned to 10 GHz may be incorporated in the antenna termination of either antenna 160 or 162 while the other antenna is terminated in a characteristic impedance, typically 75 ohms to enhance determination of the optimum coupling position of the antenna 162.

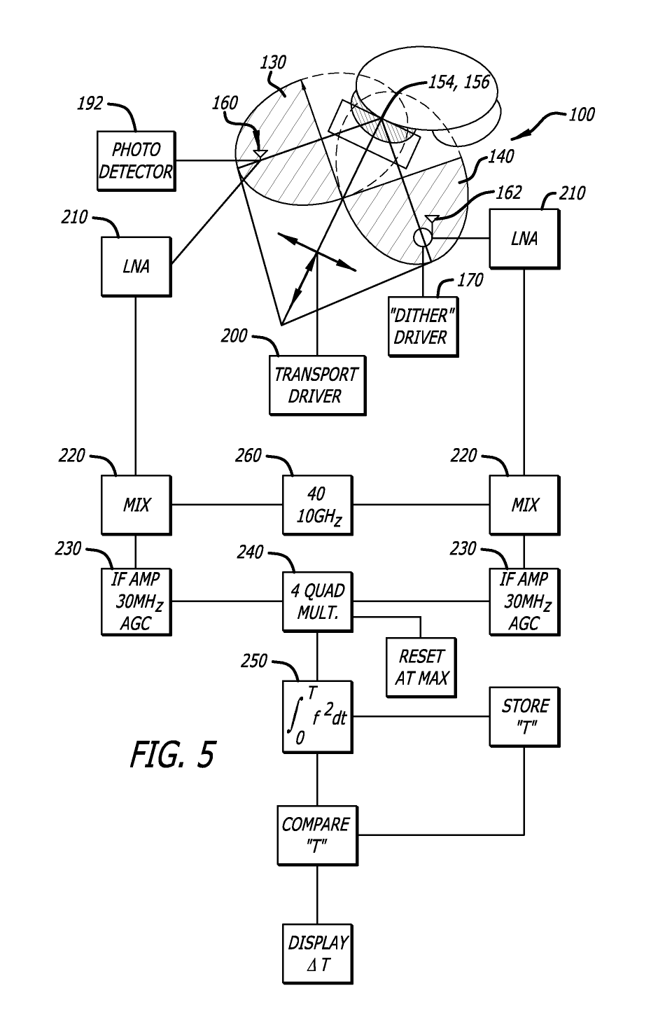

Consequently, by taking this approach, both first and second antennas 160, 162 receive signals that are comprised of coherent signals from the common focal point and incoherent signals from tissue outside the common region. A flow chart describing one approach to processing of signals of the scanning system 100 is provided in FIG. 5. Such signal processing can be accomplished using off the shelf circuitry which is commonly available.

It is also to be recognized that employing conventional approaches, the external focal point 156 of the second reflector 140 can be “dithered” or otherwise moved in the “y” direction as well as in the “x” and “z” directions. Further, a Monte Carlo method, one where the external focal point 156 of the second reflector 140 is randomly dithered or moved in multiple directions, is contemplated to achieve peak coupling of the antennas 160, 162. Upon taking this random approach, an angle associated with peak coupling is stored for subsequent positioning of the second antenna. This angle can in certain circumstances be identified for a first tissue volume examined and then used initially when beginning an examination of a second adjacent tissue volume.

Thus, as shown in FIG. 5, a scanning system 100 is electronically connected to signal processing circuitry. In this regard, the first antenna 160 associated with the first reflector 130 is immediately connected to a low noise amplifier (LNA) 210. First antenna 160 and photo detector 192 are co-located at focal point 150. Similarly, the second antenna 162 is electronically connected, to another low noise amplified (LNA) 210. The signals amplified by the LNA's are transmitted to mixers 220 where they are down-converted by oscillator 260 to a nominal 30 Mhz signal and then through intermediate frequency (IF) amplifiers 230. From there, the separate signals are further amplified in automatic gain control (AGC) circuits 230 and then multiplied in a multiplier 240, and integrated 250. It is also noted that the reflection that occurs at the skin surface of a tissue volume being examined due to the discontinuous dielectric constant of the air/skin interface is diffused since the diverging rays are out-of-focus at those sites.

Notably, the signals received by the antennas are processed in a cross-correlation detector that eliminates signals from regions outside the coherent common focal region, thereby significantly increasing the detection signal-to-noise ratio. To increase the sensitivity of the temperature measurement it is necessary to employ some focusing of the radiation into the detecting antenna. As stated, this can be accomplished by utilizing the properties of an ellipsoidal ½-reflector that has two conjugate foci.

Ellipsoidal reflectors have the property of focusing radiated energy from one focal point to the other focal point. By placing a detecting antenna within a reflector at an interior focal point, rays of energy emanating from within the tissue at an exterior focal point of the reflectors, will be concentrated at the antenna site. Further, as stated, the tissue may be “scanned” in three dimensions in a raster-scan fashion by moving the reflector/antenna assembly such as by employing a transport assembly (FIGS. 4A and 4B), and thereby the exterior focal point, to effect a volumetric survey of the entire area of tissue to be examined (i.e., breast).

Employing the signal processing approach outlined above, when the two external foci of the ellipsoidal reflectors are co-located, the signals propagating to the internal foci in the reflectors 130, 140 will be comprised of two compound signals each: f(t)+g(t) into one reflector and f(t)+h(t) into the other reflector. The signal f(t) represents the signal into each detector antenna 160, 162 from the tissue in the immediate vicinity of the common external foci 154, 156 while the signals from the tissue outside the focus vicinity generate signals into the two detector antennas of g(t) and h(t), respectively.

These signals are amplified in the low noise amplifiers (LNA) 210 and as mentioned, down-converted to a lower frequency in mixers 220 driven by a common local oscillator. In a presently contemplated approach, a 10 GHz local oscillator 260 and 30 MHz intermediate amplifier (IF) 230 can be used to convert the signal at around 10 GHz to a signal from zero to 30 MHz. The signals are amplified further in the AGC circuit 230 to nominal equal voltages. Again, the two outputs are then multiplied together in a four-quadrant linear multiplier 240 and integrated 250.

The resultant signals are then:

∫g(t)·h(t)dt+∫g(t)·f(t)dt+∫h(t)·f(t)dt+∫f(t)·f(t)dt

The first three integrals vanish since the signals g, h, and f are not coherently related and are equally likely to have instantaneous values that are (+) or (−).

The squared signal from the tissue in proximity to the external foci, however, is always positive since the functions are squared, i.e., (−)·(−)=(+) and (+)·(+)=(+).

The integral

∫{ sin(t)}² dt

evaluated between 0 and T is T/2 plus a very small oscillating term that can be ignored.

The result is that the signal from the tissue of interest at the external mutual focus point is not contaminated by signals from other tissue that contribute only background clutter noise. Such signals are eliminated for a dramatic increase in signal-to-noise ratio (SNR).

It is essential that both exterior foci 154, 156 of the two antenna assemblies coincide electromagnetically throughout the scan in order to utilize the very large SNR enhancement that comes from canceling the signal from “uninteresting” tissue. Since the radiation rays from the tissue at the nominal scan site will encounter differing dielectric materials as they travel toward each antenna at the internal focal points, the effective points of external focus will be refracted and displaced and may not coincide with the result that there is reduced cross correlation between the two signals. In order to ensure correlation, the second antenna is directed into a search mode around the position of focus of the first antenna. It is the turntable 152 to which the second antenna 162 is affixed (See FIG. 3) which facilitates ensuring this correlation.

Thus, the depth of focus of the ellipsoidal reflectors permits some misalignment of their common focus points, however, small “x” and “z” displacements of the antenna site within the second reflector 140 will permit relatively large sweeps of the external focal point of that antenna. By mounting the second antenna 162 on the rotating platform or turntable 152 in the “x”-“z” plane and rotating that antenna rapidly relative to the scan rate, e.g., 3600 rpm, it is possible to determine the optimum spatial displacement of the second antenna 162 to effect peak coincidence with the focal point of the first antenna 160.

A standard dimensional set for a breast scan is taken from a conventional mammography cassette: 24 cm×18 cm. Depth of scan is 10 cm, or more. Much of this volume is free space, however, and should be excluded from the scanning process to reduce the scan time. To do that, the surface contour of the breast must be established. A similar approach can be taken to reduce or control scanning volumes of other tissues as well.

This is accomplished by optically servoing the external focal point of the first ellipsoidal reflector 130 during a preliminary scan to track the surface contour of the tissue being scanned and store its coordinates in a look-up table. The subject tissue (i.e. breast), will be restrained in the receptacle 180, for example a brassiere that is made of cotton and contains no metallic fasteners. The cotton fabric has imprinted on it a black and white pattern of equal black and white areas for focusing purposes. The brassiere holds the breasts firmly against the chest to reduce the depth of scan required.

An opening at a base end of the first reflector 130 containing the light source 190, such as a flood lamp, is used to illuminate the brassiere white/black surface. The photo detector 192 mounted at the antenna site (internal focal point) of the first reflector 130 receives the reflected light from the brassiere surface as the scan volume is being traversed by the external focal point 154.

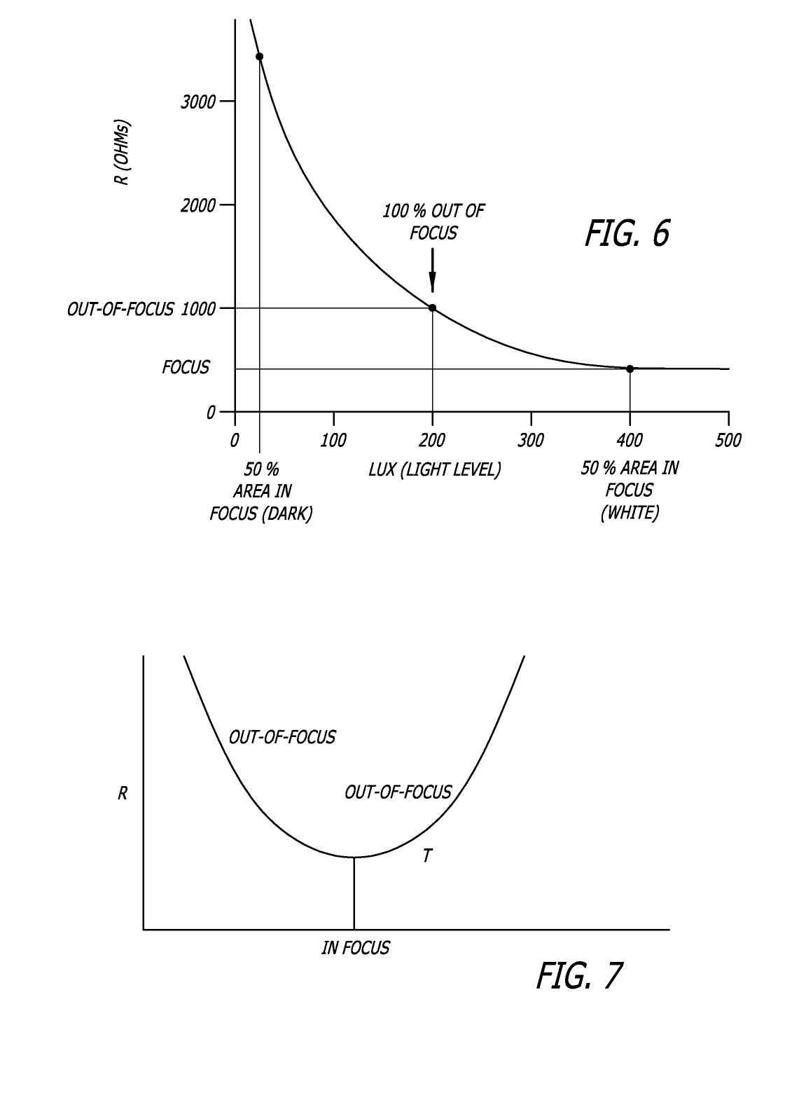

Since the resistance of the photo detector 192 is highly nonlinear in response to the light level (illuminated areas create a lower resistance than dark areas), and since the average illumination of an out-of-focus image is comprised of light contributed equally by white and dark areas, the net resistance of a focused image is much lower than that of an unfocused image (See FIGS. 6 and 7). This facilitates servoing the “z” axis of the first reflector 130 so that the lowest resistance is located and maintained throughout the “x”-“y” scan. The “x”, “y”, “z” coordinates are stored in a look-up table.

Thus, in one particular approach, the scanning procedure can involve four stages:

1) Locating and mapping the tissue (i.e., breast) surface and determining the scan boundaries,

2) Scanning the volume of the tissue while locating and storing the second antenna offset for coincidence of focus with the first antenna,

3) Finding the differential temperature of each tissue volume relative to its near-neighbors, and

4) Displaying tomographically, in false color, 3-D thermal images of the tissue (e.g. 3-D isometric, false-color format or in tomogoraphic slices).

Accordingly, in a presently contemplated approach to scanning tissue, the two approximately orthogonal reflectors 130, 140 (See FIG. 3) are mounted on a common three-axis linear actuator assembly 200 (FIGS. 4A and 4B) and aligned so that their external foci 154, 156 are coincident. The reflectors can be moved en masse to accomplish a raster-scan by the common foci in the tissue of interest. Typically the scanned volume may be 24 cm×18 cm×10 cm (a canonical measurement volume will be considered to be 1 cm³). The assembly is positioned relative to the body of the subject to effect maximum coverage of the tissue to be examined, such as a breast including the axillary region. The subject wears a special brassiere 180 that facilitates focusing the external foci 154, 156 onto the surface of the brassiere 180. The brassiere or receptacle 180 can be formed from flexible material (e.g. cotton without metallic fasteners) which retains tissue without substantial compression of the tissue. During the scan of the parallelepiped volume in “x” and “y”, the “z” axis is driven by a servo-mechanism to achieve peak focus on the brassiere 180 surface and the coordinates of each surface site are stored.

A typical scan-speed in the “x” direction will be 30 cm/sec with ½ sec allowed for step and reversal. At about 1.5 sec/line and 18 lines in the “y” direction (See FIG. 9 references A-G), the duration of the breast surface mapping and data storage will require 27 seconds.

Having determined the surface coordinates of the tissue being examined (typically reducing the scanned volume by about 60% relative to the rectilinear volume), the tissue volume is rescanned at the original 30 cm/sec rate with the scan interrupted at the tissue boundary and stepped to the next line. A typical 3-D scan will require about 1.8 minutes to completion.

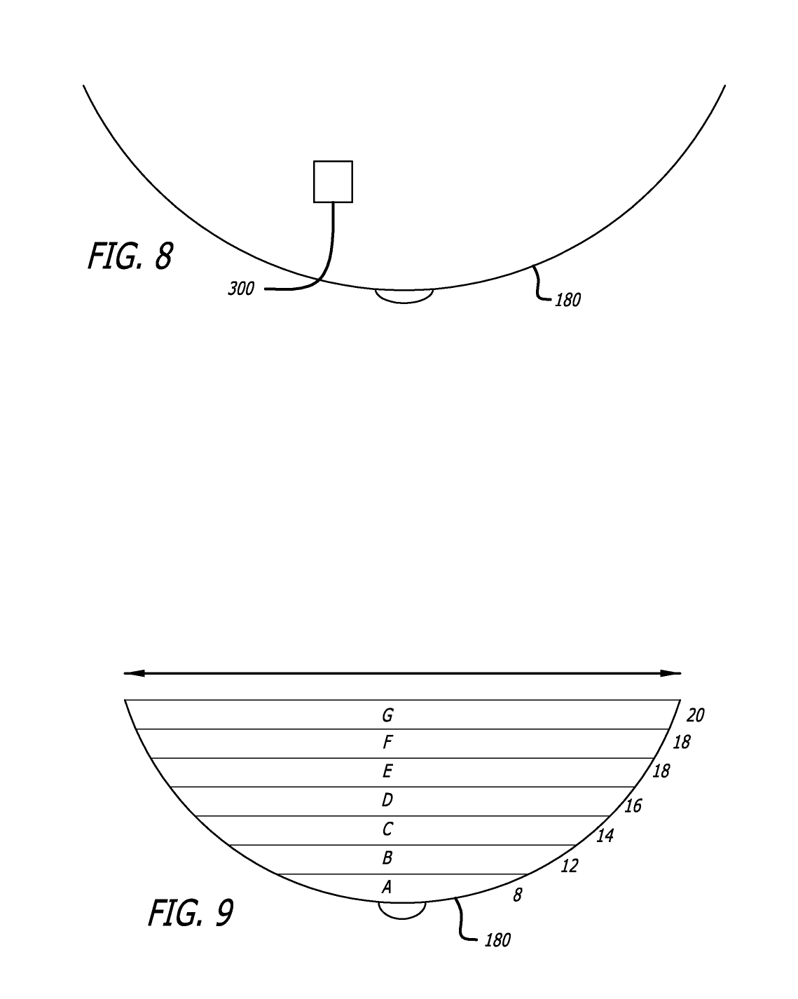

With reference to FIGS. 8 and 9, each cellular volume 300 of 1 cm³ will be traversed by the first reflector exterior focal point in about 33.3 msec. During that time, the interior antenna in the second reflector will be rotating in the “x”-“z” plane with about a 2 cm offset at a 3600 rpm rate and will make two complete revolutions during the cellular transit time. The offset of the interior antenna site will cause an offset of the external focal point. When the effective position of the exterior focal point of the second antenna is closest to the exterior focal point of the first antenna, a maximum of coupling occurs between the antennas and may be detected through impedance measurements, e.g., by terminating the second antenna by its inherent impedance and monitoring the impedance of the first antenna at 10 GHz. The angular position for peak coupling of the rotating second antenna (See FIG. 3) is noted and stored for use in a subsequent measurement scan to maximize coincidence of the two detectors.

In the measurement scan of the tissue, the stored data in the look-up table is used to correct continuously the position the second antenna during the scan of the two-reflector assembly to achieve maximum sensitivity. As each site is addressed, the second antenna displacement is adjusted for maximum coupling. As previously described, the Rayleigh-Jeans Law signal from each antenna is amplified in a LNA and mixed with a common reference local oscillator to beat the signals down to a 30 MHz IF amplifier with AGC. The signals are then multiplied together in a 4-quadrant multiplier and integrated to remove all uncorrelated “clutter” signal leaving only the signal from the common volume in the vicinity of the coincident focal point. That integrated output will rapidly increase since the integral of the squared coherent signal is integrated at 30,000 cycles/msec.

When a pre-determined integration level is reached, the integration is interrupted and the duration of integration noted as a surrogate for the temperature of that region; the shorter the integration time, the higher the temperature. That number is stored for comparison later with adjacent near-neighbor cells. For example, the integration time “T” can be stored as a surrogate for the local temperature that is compared to the six nearest-neighbor temperatures. Again, the duration of the measurement scan is about 1.8 minutes.

The procedure described above is adequate to construct a tomographic image of the breast with those sites exhibiting temperatures above the average temperature of adjacent near-neighbors highlighted through false color, or other methods.

Areas of potential interest may be revisited with a new scan to refine the resolution and temperature gradually down to about 3 mm and 0.1° C. differential temperatures. Thus, for higher resolution, the sites with elevated temperature and their immediate neighbors may by scanned again at a slower rate to increase the thermal resolution and the spatial resolution. Steps as small as 10 microns with increased integration time will allow much higher thermal and spatial resolution than is possible with the basic 1.8 minute scan. Such hot spot high-resolution re-scanning is contemplated to take approximately 20 seconds per site.

Once a lesion is detected, a biopsy can be taken or other further analysis or scan conducted to characterize the lesion. Where it is determined that the lesion is cancer or should otherwise be removed or treated, the detecting antennas are reconfigured or replaced with energy projecting antennas. Such energy projecting is then employed to treat or otherwise ablate the subject lesion. This is known as hyperthermia. Raising the lesion temperature to 113° F. is adequate to destroy it.

Accordingly, the present disclosure is intended to address passive, non-invasive tomography. The presently disclosed system thus employs a completely passive scanning of tissue, one where no radiation is generated. There is also no compression of tissue being examined and scanning can be repeated at short intervals to track identified lesions. The system is also capable of early detection of lesions (<3 mm detection threshold) and functions to minimize false negatives and false positives. Accordingly, it will be apparent from the foregoing that, while particular forms of the contemplated approaches have been illustrated and described, various modifications can be made without parting from the spirit and scope of the invention.

1. A system for detecting lesions within tissue, comprising:
a first antenna assembly including a first ellipsoidal reflector having a first exterior focal point; and
a second antenna assembly including a second ellipsoidal reflector having a second exterior focal point;
wherein the first and second exterior focal points are arranged to assume a common focal point.

2. The system of claim 1, wherein the system embodies structure with conjugate foci and defines a radiometer that passively measures electromagnetic radiation of tissues.

3. The system of claim 1, further comprising a multi-axis transport assembly, wherein the first and second ellipsoidal reflectors are half-reflectors mounted on the multi-axis transport assembly so that the first and second ellipsoidal reflectors can be translated with respect to tissue.

4. The system of claim 3, wherein the multi-axis transport assembly is configured to conduct a raster scan of tissue by moving the common focal point along target tissue.

5. The system of claim 1, further comprising a first antenna associated with a first interior focal point of the first ellipsoidal reflector and a second antenna associated with a second interior focal point of the second ellipsoidal reflector, wherein the antennas are configured to receive electromagnetic radiation of tissue emitted at a location of the common focal point.

6. The system of claim 5, wherein the second antenna is translatable with respect to the first antenna when the first and second ellipsoidal reflectors are stationary with respect to each other.

7. The system of claim 1, wherein the first and second ellipsoidal reflectors are generally positioned orthogonally with respect to each other.

8. The system of claim 1, further comprising a light source and a photocell, each being mounted within the first ellipsoidal reflector.

9. The system of claim 8, further comprising a receptacle sized and shaped to receive tissue to be scanned.

10. The system of claim 9, wherein the receptacle includes a surface having an alternating pattern of black and white unit cells.

11. The system of claim 10, wherein light energy projected by the light source onto the unit cells is detected by the photocell and analyzed to define a contour and boundary of the tissue retained in the receptacle.

12. The system of claim 11, wherein the system is configured to scan a volume of tissue contained within the contour and boundary of the receptacle and to exclude free space that is otherwise defined by an area of a conventional mammography cassette.

13. The system of claim 11, wherein a resistance of the photocell is measured and a net resistance of a focused image is located and associated with coordinates which are stored and subsequently employed to create the contour and boundary of tissue to be scanned.

14. The system of claim 1, wherein electromagnetic radiation of tissue associated with the common focal point is propagated to a first internal focal point of the first ellipsoidal reflector and to a second internal focal point of the second ellipsoidal reflector.

15. The system of claim 14, wherein the electromagnetic radiation has associated therewith first and second compound signals, each of the first and second compound signals include a first signal component representing electromagnetic radiation of tissue within an immediate vicinity of the common foci and distinct second signal components representing electromagnetic radiation of tissue outside the immediate vicinity of the common foci.

16. The system of claim 15, wherein an output associated with the first and second component signals are processed to eliminate the distinct second signal components leaving the first signal component as indicative of the tissue at the common foci.

17. The system of claim 2, wherein the system is configured to conduct a preliminary scan to map a boundary of tissue.

18. The system of claim 17, further comprising first and second antennas, wherein the system is configured to scan tissue while locating and storing an offset of the second antenna when there is a coincidence of foci with the first antenna.

19. The system of claim 18, wherein the system is configured to identify differential temperatures of a tissue volume relative to an adjacent tissue volume and to display tomographically 3-D images of tissue.

20. The system of claim 1, wherein the system is configured to identify a lesion within tissue and to transmit energy to treat the lesion.

21. A method of detecting lesions within a tissue, comprising:
configuring a first ellipsoidal antenna assembly including a first ellipsoidal reflector having a first exterior focal point and a second ellipsoidal antenna assembly including a second ellipsoidal reflector having a second exterior focal point adjacent the tissue;
aligning the first and second exterior focal points within a unit cell of tissue; and
detecting electromagnetic energy within the unit cell.

22. The method of claim 21, further comprising placing the tissue within a receptacle and identifying and determining a boundary of the tissue to be scanned.

23. The method of claim 22, further comprising providing the first ellipsoidal reflector with a light source and a photodetector, projecting light onto the receptacle, and measuring a resistance of the photodetector as a function of light reflected from the receptacle.

24. The method of claim 22, further comprising providing the first ellipsoidal reflector with a first antenna and providing the second ellipsoidal reflector with a second antenna, the second antenna being translatable with respect to the first antenna when the ellipsoidal reflectors are held stationary.

25. The method of claim 24, further comprising scanning a volume of the tissue while locating and storing an offset for coincidence of focus of the second antenna with the first antenna.

26. The method of claim 25, further comprising finding a differential temperature of each volume of tissue relative to adjacent tissue volume.

27. The method of claim 26, further comprising displaying tomographically 3-D images of the tissue volumes.

28. The method of claim 25, further comprising employing the offset information in subsequently scanning tissue.

29. The method of claim 28, further comprising conducting a subsequent scan of areas of tissue identified as having a higher temperature than adjacent tissue.

30. The method of claim 21, further comprising directing energy at a detected lesion to treat the detected lesion.
